(19) United States
(12) Patent Application Publication
(10) Pub. No.: US 2010/0081622 A1
(43) Pub. Date: Apr. 1, 2010

(54) Reduction of parathyroid hormone levels

(75) Inventors: Bolette Hartmann, Hellerup (DK); Dennis B. Henriksen, Allerod (DK)

Correspondence Address: EDWARDS ANGELL PALMER & DODGE LLP, P.O. BOX 55874, BOSTON, MA, 02205, US

(73) Assignee: Sanos Bioscience A/S

(21) Appl. No.: 12/586,875

(22) Filed: Sep. 28, 2009

Related U.S. Application Data
(60) Provisional application No. 61/194,627, filed on Sep. 29, 2008.

(51) Int. Cl. A61K 38/22; A61P 5/18

(52) U.S. Cl. 514/12

(57) ABSTRACT

A method of acutely reducing the plasma level of PTH of a patient having an elevated PTH comprising the administration of a pharmaceutical composition comprising a GLP-2, or a variant, an analogue, or derivative of GLP-2 having the ability to bind and activate a GLP-2 receptor such as GLP-2 1-34.

The present invention relates to the use of GLP-2 and variants; analogues and derivatives thereof (GLP-2 compounds) for reducing serum levels of parathyroid hormone (PTH). In an alternative aspect, the invention relates to the use of GLP-2 compounds in treating dietary calcium malabsorption, e.g. as a consequence of gastric bypass surgery.

WO 02/22151 and WO 02/24214 described the use of GLP-2 compounds thereof for reducing the rate of resorption of bone by reducing the bone resorbing activity of osteoclasts. It was indicated that such treatment might include mitigation of bone resorption caused by excessive levels of parathyroid hormone (PTH)—hyperparathyroidism. The basis for that use of GLP-2 was a finding that the rate of bone resorption as measured by serum levels of bone resorption derived peptides fell almost immediately on administration of GLP-2. This of course was only treatment of hyperparathyroidism in the sense of treating one of the consequential symptoms or sequelae thereof.

The bony skeleton serves as a store for calcium to and from which calcium may be moved so as to maintain serum calcium homeostasis through the day and night. Calcium inputs are derived from diet following meals at spaced intervals. Osteoblasts deposit calcium in bone and osteoclasts resorb calcium from bone and their respective rates of operation in this regard are controlled to maintain calcium homeostasis via a complex hormone driven mechanism in which PTH is a major component.

Low serum calcium stimulates release of PTH by the parathyroid glands leading to stimulation of osteoclasts to resorb calcium from the skeleton. A rise in calcium level acts to reduce PTH secretion. Prolonged reduction of calcium levels can lead to chronically raised PTH levels (hyperparathyroidism (HPT)), leading to undesirable loss of bone mass. Equally chronically raised PTH levels arising from other causes can lead to such bone loss.

HPT is characterized by increased secretion of parathyroid hormone (PTH). HPT is divided into 3 subtypes: primary, secondary and tertiary.

Primary HPT (pHPT) is generally the consequence of an autonomous adenoma of the parathyroid gland(s) or, in rare cases, parathyroid carcinoma. It is an endocrine disorder characterized by incompletely regulated, chronic, excessive secretion of PTH from one or more parathyroid glands. Parathyroidectomy (PTX) is currently the only curative treatment for pHPT. There are currently no non-surgical therapies approved for use in pHPT, although bisphosphonates are used in some patients, in an effort to control serum calcium levels. Calcimimetics (molecules that activate the calcium-sensing receptor and inhibit PTH secretion) have been suggested as an alternative to PTX. Calcimemetics may result in normalized serum calcium whilst only modestly reducing serum PTH levels.

Secondary HPT (sHPT) is characterized by an increase in PTH secretion as a compensatory response to reduced serum calcium levels. It is commonly seen in patients with chronic kidney disease (CKD). sHPT develops early in CKD when serum calcium and phosphate are still within normal limits and is present virtually in all patients with end-stage renal disease (ESRD). As CKD progress, calcitriol (the active form of vitamin D) production by the kidneys decreases, and serum PTH increases. Several treatment options have been proposed and are used in daily practice. In ESRD patients, calcimimetics were shown to simultaneously reduce PTH, calcium and phosphate. Treatment with calcitriol lowers PTH but often raises calcium and phosphate levels and was shown to induce moderate to marked aortic calcification in rats.

Elevated PTH and sHPT is also common following gastric bypass (GBP) in obese patients. In a recent study, 5% of the patients had sHPT immediately postoperatively and this ratio increased to 21% at one year. This sHPT seems to be driven by calcium and vitamin D malabsorption. However, sHPT was also seen in short-limb GBP (<100 cm) with vitamin levels >30 ng/ml suggesting selective calcium malabsorption.

In addition to CKD and GBP, sHPT is observed in metastatic prostate cancer. Osteoblastic metastases cause an increased deposition of calcium and phosphate in bone inducing hypocalcaemia and elevated PTH levels. It has been proposed that suppressing serum PTH in advanced prostate cancer may reduce morbidity and mortality.

Tertiary HPT (tHPT) is defined as persistent HPT after prolonged stimulus to PTH secretion (i.e. sHPT) in which serum calcium increases. This can occur prior to or following renal transplantation. PTX is currently used in the treatment of tHPT. However, several reports indicate that up to 29% of patients with tHPT may have disease limited to only one or two glands and resection of only the enlarged glands has been proposed. As an alternative to surgery, treatment of tHPT with Cinacalcet has been reported with a significant reduction in serum PTH (21.8% after 10 weeks of treatment) being observed.

Elevation of PTH has also been found to be associated with obesity.

It has now been observed that administration of GLP-2 not only reduces levels of bone resorption markers but also causes a similarly rapid decrease in serum PTH.

Accordingly, in a first aspect, the invention provides a pharmaceutical composition comprising a GLP-2, or a variant, an analogue, or derivative of GLP-2 having the ability to bind and activate a GLP-2 receptor, for use in treatment by reducing an elevated level of PTH. The elevated level of PTH may be primary, secondary, or tertiary hyperparathyroidism and in particular may be secondary to gastrointestinal bypass surgery or to dietary calcium malabsorption or may be hyperparathyroidism linked to obesity.

The composition may comprise as one or as the only active component GLP-2 1-34.

The invention includes a method of acutely reducing the plasma level of PTH of a patient having an elevated PTH comprising the administration of a pharmaceutical composition comprising a GLP-2, or a variant, an analogue, or derivative of GLP-2 having the ability to bind and activate a GLP-2 receptor.

This discovery opens up the possibility of treating patients suffering from elevation of PTH even if they show no sign of increased bone resorption or net loss of bone mass. This finding is quite unexpected especially in that reduction in bone resorption would be expected to lower serum calcium and low serum calcium is a trigger for release of PTH.

Said GLP-2 may be any of the forms of GLP-2 discussed below including variants, analogues and derivatives of naturally occurring GLP-2, including GLP-2 (1-34), especially human GLP-2 (1-34).

GLP-2 derivatives for use in the invention may include salts, esters or amides.

One or more additional therapeutic agents can be administered in conjunction with the compositions of the invention.

Also contemplated by the invention are methods of determining the effectiveness of PTH lowering treatment with a GLP-2 in a patient comprising:

(a) determining the level of PTH from a first patient tissue sample prior to said treatment and a second patient tissue sample after said treatment;
(b) comparing said PTH levels in said tissue samples, wherein a decrease in said level in said second tissue sample indicates effective treatment.

Also contemplated by the invention are methods of determining the effectiveness of therapeutic or prophylactic treatment by administration of a composition of the invention in a patient comprising:

(a) determining the level of PTH from a first patient tissue sample prior to said administration and a second patient tissue sample after said administration;

(b) comparing said PTH levels in said tissue samples, wherein a decrease in said level in said second tissue sample indicates effective therapeutic treatment and prevention of an increase in said level in said second tissue sample indicates effective prophylactic treatment.

### DEFINITIONS

As used herein, the “GLP-2” includes naturally occurring GLP-2 peptides from various species.

As used herein, the terms “variant” or “variants” refer to variations of the amino acid sequence of a GLP-2 Encompassed within these terms are amino acid substitutions, additions, or deletions of natural GLP-2 amino acid sequences. Also encompassed are chemically modified natural and synthetic GLP-2 peptides.

A polypeptide that has a similar amino acid sequence is preferred. This refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a GLP-2 polypeptide or a fragment of a GLP-2 polypeptide described herein; (b) a polypeptide encoded by a nucleotide sequence that hybridizes under stringent conditions to a nucleotide sequence encoding a GLP-2 polypeptide or a fragment of a GLP-2 polypeptide described herein of at least 10 amino acid residues, at least 15 amino acid residues, at least 20 amino acid residues, at least 25 amino acid residues, or at least 30 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a GLP-2 polypeptide or a fragment of a GLP-2 polypeptide described herein. A polypeptide with similar structure to a GLP-2 polypeptide or a fragment of a GLP-2 polypeptide described herein refers to a polypeptide that has a similar secondary, tertiary or quaternary structure of a GLP-2 polypeptide or a fragment of a GLP-2 polypeptide described herein. The structure of a polypeptide can be determined using methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (e.g., http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “analogue” encompasses a polypeptide that possesses a similar or identical function to a GLP-2 polypeptide or a fragment of a GLP-2 polypeptide, but does not necessarily comprise a similar or identical amino acid sequence of a GLP-2 polypeptide or a fragment of a GLP-2 polypeptide. The term “analogue” also encompasses non-peptide molecules having the ability to bind and activate a GLP-2 receptor.

As used herein, the term “fragment” or “fragments” as used herein refers to a peptide or polypeptide having an amino acid sequence of at least 10 contiguous amino acid residues, at least 15 contiguous amino acid residues, at least 20 contiguous amino acid residues, at least 25 contiguous amino acid residues, or at least 30 contiguous amino acid residues of the amino acid sequence of a GLP-2 polypeptide.

GLP-2, variants, analogues and derivatives are referred to collectively herein as “GLP-2 compounds”.

As used herein, the term “patient” is an animal, such as, but not limited to, a cow, monkey, horse, sheep, pig, chicken, turkey, quail, cat, dog, mouse, rat, rabbit, and guinea pig, and is more preferably a mammal, and most preferably a human.

As used herein, the phrase “pharmaceutically acceptable” refers to an agent that does not interfere with the effectiveness of the biological activity of an active ingredient, and which may be approved by a government regulatory agency, or is listed in the U.S. Pharmacopeia or other generally recognized pharmacopeia for use in animals, and more particularly for use in humans. Accordingly, suitable pharmaceutically acceptable carriers include agents that do not interfere with the effectiveness of a pharmaceutical composition.

As used herein, the phrase “pharmaceutically acceptable salts” refers to salts prepared from pharmaceutically acceptable, preferably non-toxic, acids and bases, including inorganic and organic acids and bases, including but not limited to, sulphuric, citric, maleic, acetic, oxalic, hydrochloride, hydro bromide, hydro iodide, nitrate, sulphate, bisulphite, phosphate, acid phosphate, iso-nicotinoate, acetate, lactate, salicylate, citrate, acid citrate, tartrate, oleate, tannate, pantothenate, bi-tartrate, ascorbate, succinate, maleate, gentisinate, fumarate, gluconate, glucaronate, saccharate, formate, benzoate, glutamate, methanesulphonate, ethanesulphonate, benzenesulphonate, p-toluenesulphonate and pamoate (i.e., 1,1′-methylene-bis-(2-hydroxy-3-naphthoate)) salts. Pharmaceutically acceptable salts include those formed with free amino groups such as, but not limited to, those derived from hydrochloric, phosphoric, acetic, oxalic, and tartaric acids. Pharmaceutically acceptable salts also include those formed with free carboxyl groups such as, but not limited to, those derived from sodium, potassium, ammonium, sodium lithium, calcium, ferric hydroxides, isopropylamine, triethylamine, 2-ethylamino ethanol, histidine, and procaine.

As used herein, the term “carrier” refers to a diluent, adjuvant, excipient, or vehicle. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions.

As used herein, the term “mineral” refers to a substance, preferably a natural substance, that contain calcium, magnesium or phosphorus. Illustrative nutrients and minerals include beef bone, fish bone, calcium phosphate, egg shells, sea shells, oyster shells, calcium carbonate, calcium chloride, calcium lactate, calcium gluconate and calcium citrate.

As used herein, the term “biological sample” is broadly defined to include any cell, tissue, organ or multicellular organism. A biological sample can be derived, for example, from cells or tissue cultures in vitro. Alternatively, a biological sample can be derived from a living organism or from a population of single cell organisms. Preferably, the biological sample is live tissue.

As used herein, the phrase “isolated polypeptide or peptide” refers to a polypeptide or peptide that is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein, peptide, or fragment thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest. In preferred embodiments, purified or isolated preparations will lack any contaminating proteins from the same animal from which the protein is normally produced, as can be accomplished by recombinant expression of, for example, a human protein in a non-human cell.

As used herein, the phrase “isolated nucleic acid molecule” refers to a nucleic acid molecule which is separated from other nucleic acid molecules which are in the natural source of the nucleic acid molecule. Preferably, an isolated nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. In other embodiments, the isolated nucleic acid is free of intron sequences. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an isolated nucleic acid molecule, such as a cDNA molecule, can be substantially free of other cellular material, or culture medium when produced by recombinant techniques, or substantially free of chemical precursors or other chemicals when chemically synthesized. In one embodiment, the nucleic acid molecules of the invention comprise a contiguous open reading frame encoding a polypeptide of the invention.

As used herein, the phrase “hybridises under stringent conditions” is intended to describe conditions for hybridization and washing under which nucleotide sequences at least 60% (65%, 70%, 75%, 80%, or preferably 85% or more) identical to each other typically remain hybridized to each other. Such stringent conditions are known to those skilled in the art and can be found in Current Protocols in Molecular Biology, John Wiley & Sons, N.Y. (1989), 6.3.1-6.3.6, which describes aqueous and non-aqueous methods, either of which can be used. Another preferred, non-limiting example of stringent hybridization conditions are hybridization in 6× sodium chloride/sodium citrate (SSC) at about 45° C., followed by one or more washes in 2.0×SSC at 50° C. (low stringency) or 0.2×SSC, 0.1% SDS at 50-65° C. (high stringency). Another preferred example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions are hybridization in 6× sodium chloride/sodium citrate (SSC) at about 45° C., followed by one or more washes in 0.2×SSC, 0.1% SDS at 60° C. Preferably, stringent hybridization conditions are hybridization in 6× sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C. In one embodiment, an isolated nucleic acid molecule of the invention that hybridizes under stringent conditions to the sequence of the GLP-2 nucleic acid, or a complement thereof, corresponds to a naturally-occurring nucleic acid molecule. As used herein, a “naturally occurring” nucleic acid molecule refers to an RNA or DNA molecule having a nucleotide sequence that occurs in nature (e.g., encoding a natural protein).

### GLP-2

GLP-2 is a peptide produced as one of several expression products of the glucagon gene. Mammalian GLP-2 is generally a 33 amino acid peptide (GLP-2 1-33). References to longer GLP-2 peptides such as GLP-2 1-34 herein refer primarily to GLP-2 1-33 extended in the C-terminal direction by the amino acids that follow the GLP-2 sequence in the coding of the glucagon gene in the appropriate species.

GLP-2 is highly conserved between species. By way of example, the following are the sequences of GLP-2 in the species indicated, set within the context of the sequence of proglucagon, where residue 126 of proglucagon corresponds to residue 1 of GLP-2:

Human
126
His-Ala-Asp-Gly-Ser-Phe-Ser-Asp-Glu-Met-Asn-Thr-
138 144
Ile-Leu-Asp-Asn-Leu-Ala-Ala-Arg-Asp-Phe-Ile-Asn-
152 160
Trp-Leu-Ile-Gln-Thr-Lys-Ile-Thr-Asp-Arg-Lys
Porcine
His-Ala-Asp-Gly-Ser-Phe-Ser-Asp-Glu-Met-Asn-Thr-
Val-Leu-Asp-Asn-Leu-Ala-Thr-Arg-Asp-Phe-Ile-Asn-
Trp-Leu-Leu-His-Thr-Lys-Ile-Thr-Asp-Ser-Leu
Rat
His-Ala-Asp-Gly-Ser-Phe-Ser-Asp-Glu-Met-Asn-Thr-
Ile-Leu-Asp-Asn-Leu-Ala-Thr-Arg-Asp-Phe-Ile-Asn-
Trp-Leu-Ile-Gln-Thr-Lys-Ile-Thr-Asp-Lys-Lys
Hamster
His-Ala-Asp-Gly-Ser-Phe-Ser-Asp-Glu-Met-Asn-Thr-
Ile-Leu-Asp-Ser-Leu-Ala-Thr-Arg-Asp-Phe-Ile-Asn-
Trp-Leu-Ile-Gln-Thr-Lys-Ile-Thr-Asp-Lys-Lys
Anglerfish
His-Ala-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp-Arg-Lys
Guinea Pig
His-Ala-Asp-Gly-Ser-Phe-Ser-Asp-Glu-Met-Asn-Thr-
Ile-Leu-Asp-Asn-Leu-Ala-Thr-Arg-Asp-Phe-Ile-Asn-
Trp-Leu-Ile-Gln-Thr-Lys-Ile-Thr-Asp-Arg-Lys
Bovine
His-Ala-Asp-Gly-Ser-Phe-Ser-Asp-Glu-Met-Asn-Thr-
Val-Leu-Asp-Ser-Leu-Ala-Thr-Arg-Asp-Phe-Ile-Asn-
Trp-Leu-Leu-Gln-Thr-Lys-Ile-Thr-Asp-Arg-Lys
Mouse
His-Ala-Asp-Gly-Ser-Phe-Ser-Asp-Glu-Met-Ser-Thr-
Ile-Leu-Asp-Asn-Leu-Ala-Thr-Arg-Asp-Phe-Ile-Asn-
Trp-Leu-Ile-Gln-Thr-Lys-Ile-Thr-Asp-Arg-Lys
Dog
His-Ala-Asp-Gly-Ser-Phe-Ser-Asp-Glu-Met-Asn-Thr-
Val-Leu-Asp-Thr-Leu-Ala-Thr-Arg-Asp-Phe-Ile-Asn-
Trp-Leu-Leu-Gln-Thr-Lys-Ile-Thr-Asp-Arg-Lys
Degu
His-Ala-Asp-Gly-Ser-Phe-Ser-Asp-Glu-Met-Asn-Thr-
Val-Leu-Asp-His-Leu-Ala-Thr-Lys-Asp-Phe-Ile-Asn-
Trp-Leu-Ile-Gln-Thr-Lys-Ile-Thr-Asp-Arg-Lys
Salamander
His-Ala-Asp-Gly-Ser-Phe-Thr-Ser-Asp-Ile-Asn-Lys-
Val-Leu-Asp-Thr-Ile-Ala-Ala-Lys-Glu-Phe-Leu-Asn-
Trp-Leu-Ile-Ser-Thr-Lys-Val-Thr-Glu-Arg-Lys

Human GLP-2 1-34 is accordingly:

His-Ala-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp-Arg

The biological role of GLP-2 remains unclear. As well as its effect on the rate of bone resorption discussed above, it has been found to have intestinotrophic effects.

The invention may employ all natural forms of GLP-2 as well as variants, analogs and derivatives thereof.

Compared to a naturally occurring GLP-2 molecule, a variant thereof for use in the invention may contain additional amino acid residues, or have amino acids deleted from it or substituted in it. A GLP-2 analogue or variant may have enhanced activity compared to native human GLP-2. For example, such GLP-2 variants and analogues can exhibit enhanced serum stability, enhanced receptor binding, or enhanced signal transducing activity. Amino acid modifications, substitutions, additions, or truncations that render a GLP-2 peptide resistant to oxidation or degradation may be used. In a preferred embodiment, the GLP-2 variants are derived from human or rat GLP-2 sequences.

Molecules contemplated as GLP-2 compounds, in accordance with the present invention are known in the art. For example, U.S. Pat. No. 5,990,077, discloses forms of GLP-2 and the pharmaceutically acceptable acid salts thereof, that conform to the general formula:

R1-[Y]m-His-Ala-Asp-Gly-Ser-Phe-Ser-Asp-Glu-Met-
Asn-Thr-aa1-Leu-Ala-aa2-Leu-Ala-aa3-Arg-Asp-Phe-
Ile-Asn-Trp-Leu-aa4-aa5-Thr-Lys-Ile-Thr-Asp-[X]-
n-R2.

Variants can be prepared using standard solid-phase techniques for the synthesis of peptides. As is generally known, peptides of the requisite length can be prepared using commercially available equipment and reagents following the manufacturers' instructions for blocking interfering groups, protecting the amino acid to be reacted, coupling, deprotection, and capping of unreacted residues. Suitable equipment can be obtained, for example, from Applied BioSystems in Foster City, Calif., or Biosearch Corporation in San Raphael, Calif. It is also possible to obtain fragments of GLP-2, by fragmenting the naturally occurring amino acid sequence, using, for example, a proteolytic enzyme. Further, it is possible to obtain the desired fragments of the GLP-2 through the use of recombinant DNA technology. The basic steps in recombinant production are:

a) isolating a natural DNA sequence encoding GLP-2 or constructing a synthetic or semi-synthetic DNA coding sequence for GLP-2,
b) placing the coding sequence into an expression vector in a manner suitable for expressing proteins either alone or as a fusion proteins,
c) transforming an appropriate eukaryotic or prokaryotic host cell with the expression vector,
d) culturing the transformed host cell under conditions that will permit expression of a GLP-2 intermediate, and
e) recovering and purifying the recombinantly produced protein.

The GLP-2 compound may be a GLP-2 variant having enhanced resistance to degradation as compared to native GLP-2. Enhanced resistance to degradation can result in longer bioavailability. In a specific embodiment, the GLP-2 variant demonstrates both enhanced insulin-release stimulating activity and enhanced stability.

Examples of GLP-2 variants are found in U.S. Pat. Nos. 5,990,077 and 6,184,201, and include the following:

| | |
|---|---|
| 1) | His-Ala-Asp-Gly-Ser-Phe-Ser-Asp-Glu-Met-Asn- |
| | Thr-Ile-Leu-Asp-Asn-Leu-Ala-Thr-Arg-Asp-Phe- |
| | Ile-Asn-Trp-Leu-Ile-Gln-Thr-Lys-Ile-Thr-Asp. |
| 2) | R1-[Y]m-His-Ala-Asp-Gly-Ser-Phe-Ser-Asp-Glu- |
| | Met-Asn-Thr-aa1-Leu-Asp-aa2-Leu-Ala-aa3-Arg- |
| | Asp-Phe-Ile-Asn-Trp-Leu-aa4-aa5-Thr-Lys-Ile- |
| | Thr-Asp-[X]n-R2. |

wherein:
aa1 is a neutral, polar, large and nonaromatic amino acid residue;
aa2 is a neutral and polar amino acid residue;
aa3 is a neutral amino acid residue;
aa4 is a neutral, polar, large and nonaromatic amino acid residue;
aa5 is a neutral or basic amino acid residue;

### X is Arg, Lys, Arg-Lys or Lys-Lys;

### Y is Arg or Arg-Arg;

m is 0 or 1;
n is 0 or 1;
R1 is H or an N-terminal blocking group; and
R2 is OH or a C-terminal blocking group.

| | |
|---|---|
| 3) | R1-[Y]m-His-Ala-Asp-Gly-Ser-Phe-Ser-Asp-Glu- |
| | Met-Asn-Thr-aa1-Leu-Asp-aa2-Leu-Ala-aa3-Arg- |
| | Asp-Phe-Ile-Asn-Trp-Leu-aa4-aa5-Thr-Lys-Ile- |
| | Thr-Asp-[X]n-R2 |

wherein:
aa1 is Ile or Val;
aa2 is Asn or Ser;
aa3 is Ala or Thr;
aa4 is Ile or Leu;
aa5 is Gln or His;

### X is Arg, Lys, Arg-Lys or Lys-Lys;

### Y is Arg or Arg-Arg;

m is 0 or 1;
n is 0 or 1;
R1 is H or an N-terminal blocking group; and
R2 is OH or a C-terminal blocking group.

| | |
|---|---|
| 4) | R1-(Y1)m-X1-X2-X3-X4-Ser5-Phe6-Ser7-Asp8-(P1)- |
| | Leu14-Asp15-Asn16-Leu17-Ala18-X19-X20-Asp21- |
| | Phe22-(P2)-Trp25-Leu26-Ile27-Gln28-Thr29-Lys30- |
| | (P3)-(Y2)n-R2, |

wherein
X1 is His or Tyr
X2 is Ala or an Ala-replacement amino acid conferring on said analog resistance to DPP-IV enzyme;
X3 is Pro, HPro, Asp or Glu;
X4 is Gly or Ala;
P1 is Glu-X10-Asn-Thr-Ile or Tyr-Ser-Lys-Tyr;
X10 is Met or an oxidatively stable Met-replacement amino acid;
X19 is Ala or Thr;
X20 is Arg, Lys, His or Ala;
P2 is Ile-Asn, Ile-Ala or Val-Gln;
P3 is a covalent bond, or is Ile, Ile-Thr or Ile-Thr-Asn;
R1 is H or an N-terminal blocking group;
R2 is OH or a C-terminal blocking group;
Y1 is one or two basic amino acids selected from the group Arg, Lys, and His;
Y2 is one or two basic amino acids selected from the group Arg, Lys, and His; and
m and n, independently, are 0 or 1; and
   wherein at least one of X1, X2, X3, X4, P1, X10, X19, X20, P2 and P3 is other than a wild type, mammalian GLP-2 residue. These and other GLP-2 variants may be employed in the invention.

Unless otherwise specified, the term GLP-2 includes collectively the various synthetically or recombinantly produced forms of GLP-2, particularly the mammalian forms, e.g., rat GLP-2, ox GLP-2, porcine GLP-2, bovine GLP-2, guinea pig GLP-2, hamster GLP-2 and human GLP-2, the sequences of which have been reported by many authors including Chu et all in Central Nervous System Agents in Medicinal Chemistry, 2006, 6, 27-57. Taking into account the significant sequence homology among these GLP-2 species, the present invention embraces the use as reducer of PTH levels of those forms of GLP-2 and the pharmaceutically acceptable acid salts thereof, that conform to the general formula represented below:

R1-(Y1)m-X1-X2-X3-X4-X5-X6-X7-X8-X9-X10-X11-X12-
X13-X14-X15-X16-X17-X18-X19-X20-X21-X22-X23-X24-
X25-X26-X27-X28-X29-X30-X31-X32-X33-(Y2)n-R2

wherein:
R1 is H or an N-terminal blocking group;
(Y1) is one or two basic amino acids selected from the group Arg, Lys, and His;

### X1 is X0, His or Tyr;

X2 is X0, Ala, Leu, Cys, Glu, Arg, Trp, Tyr, DhPr, D-Pro, D-Ala, Gly, Val, Lys, Ile, Trp, PO₃-Tyr, Cys, or an Ala- replacement amino acid which confers on the analog or salt resistance to cleavage by human DPP-IV enzyme; (preferably X2 is X0, Ala, Leu, Cys, Glu, Arg, Trp, Tyr, or an Ala- replacement amino acid which confers on the analog or salt resistance to cleavage by human DPP-IV enzyme;)

### X3 is X0, Pro, HPro, Asp or Glu;

### X4 is X0, Gly or Ala;

### X5 is Ser or Xd;

### X 6 is Phe;

### X7 is Ser or Xd;

### X8 is Asp;

### X9 is Glu or Tyr;

X10 is Met or oxidisable stable Met analogue, Val, Ile, Asn, Glu, Gln, Tyr, Phe, Leu, Nle, Ala, Gly, or Ser; (preferably X10 is Met or oxidisable stable Met analogue, or Ser;)

### X11 is Asn or Lys;

### X12 is Thr or Tyr;

X13 is Ile, Val or a neutral, polar, large and nonaromatic amino acid residue;

### X14 is Leu;

### X15 is Asp or Xa;

X16 is Asn, Ser or a neutral and polar amino acid residue;

### X17 is Leu;

### X18 is Ala;

X19 is Ala, Thr or a neutral amino acid residue;

### X20 is Arg, Lys, His or Ala;

### X21 is Asp;

### X22 is Phe or Xb;

### X23 is Ile or Val;

### X24 is Asn, Gln or Ala;

### X25 is Trp;

### X26 is Leu;

X27 is Ile, Leu or a neutral, polar, large and nonaromatic amino acid residue;
X28 is Gln, His or a neutral or basic amino acid residue;

### X29 is Thr or Xc;

### X30 is Lys;

### X31 is Ile or Arg;

### X32 is Thr, Lys or Xc;

### X33 is Asp, Asn, His or Xa;

X0 is an amino acid deletion;
Xa is any amino acid other than Asp;
Xb is any amino acid other than Phe;
Xc is any aminoacid other than Thr;
Xd is any amino acid other than Ser;
Y2 is one or two basic amino acids selected from the group Arg, Lys, and His;
m and n are independently 0 or 1 and wherein at least one of X1-X2-X3-X4-X5-X6-X7-X8-X9-X10-X11-X12-X13-X14-X15-X16-X17-X18-X19-X20-X21-X22-X23-X24-X25-X26-X27-X28-X29-X30-X31-X32-X33 is other than wild type, mammalian GLP-2 residue, and
R2 is OH or a C-terminal blocking group.

In particular embodiments of the invention, the GLP-2 conforms to the sequence shown below:

R1-[Y1]-His-Ala-Asp-Gly-Ser-Phe-Ser-Asp-Glu-Met-
Asn-Thr-Ile-Leu-Asp-Asn-Leu-Ala-X19-Arg-Asp-Phe-
Ile-Asn-Trp-Leu-Ile-Gln-Thr-Lys-Ile-Thr-Asp-[Y2]n-
R2

wherein X19, Y1, Y2, n, R1 and R2 are as defined above
In a specific embodiment of the invention, GLP-2 has the sequence illustrated below:

His-Ala-Asp-Gly-Ser-Phe-Ser-Asp-Glu-Met-Asn-Thr-
Ile-Leu-Asp-Asn-Leu-Ala-Thr-Arg-Asp-Phe-Ile-Asn-
Trp-Leu-Ile-Gln-Thr-Lys-Ile-Thr-Asp

The “blocking groups” represented by R1 and R2 are chemical groups that are routinely used to confer biochemical stability and resistance to digestion by exopeptidase. Suitable N-terminal protecting, groups include, for example, C₁₋₅ alkanoyl groups such as acetyl. Also suitable as N-terminal protecting groups are amino acid analogues lacking the amino function. Suitable C-terminal protecting groups include groups which form ketones or amides at the carbon atom of the C-terminal carboxyl, or groups which form esters at the oxygen atom of the carboxyl. Ketone and ester-forming groups include alkyl groups, particularly branched or unbranched C₁₋₅ alkyl groups, e.g. methyl, ethyl and propyl groups, while amide-forming groups include amino functions such as primary amine, or alkylamino functions, e.g. mono-C₁₋₅ alkylamino and di-C₁₋₅ alkylamino groups such as methylamino, ethylamino, dimethylamino, diethylamino, methylethylamino and the like. Amino acid analogues are also suitable for protecting the C-terminal end of the present compounds, for example, decarboxylated amino acid analogues such as agmatine (decarboxylated arginine).

The particular form of GLP-2 selected for use in the invention can be prepared by a variety of techniques well known for generating peptide products. As described by Pedersen et al in Regulatory Peptides 146 (2008) 310-320, porcine GLP-2 isolation and purification is achieved from ileal mucosa, obtained from anesthetized pigs. The frozen tissue was crushed and extracted at neutral pH by boiling in demineralized water followed by homogenization and centrifugation at 4° C. Followed by HPLC purification with the aid of antibody raised against synthetic proglucagon 126-159, to monitor work-up. As an alternative to GLP-2 extraction, those forms of GLP-2 that incorporate only L-amino acids can be produced reproducibly and in commercial quantities by application of recombinant DNA technology. For this purpose, DNA coding for the desired form of GLP-2 is incorporated expressibly in a microbial e.g. yeast, or other cellular host, which is then cultured under conditions appropriate for GLP-2 expression. A variety of gene expression systems have been adapted for this purpose, and typically drive expression of the desired gene from expression controls used naturally by the chosen host. Because GLP-2 does not require post translational glycosylation for its activity, its production may most conveniently be achieved in bacterial hosts such as E. coli. For such production, DNA coding for the selected GLP-2 may usefully be placed under expression controls of the lac, trp or PL genes of E. coli. As an alternative to expression of DNA coding for the GLP-2 per se, the host can be adapted to express GLP-2 as a fusion protein it which the GLP-2 is linked releasably to a carrier protein that facilitates isolation and stability of the expression product.

In an approach universally applicable to the production of a selected GLP-2 compound, and one used necessarily to produce GLP-2 forms that incorporate non-genetically encoded amino acids and N- and C-terminally derivatised forms, the-well established techniques of automated peptide synthesis are employed, general descriptions of which appear, for example, in J. M. Stewart and J. D. Young, Solid Phase Peptide Synthesis, 2nd Edition, 1984, Pierce Chemical Company, Rockford, Ill.; and in M. Bodanszky and A. Bodanszky, The Practice of Peptide Synthesis, 1984, Springer-Verlag, New York; Applied Biosystems 430A Users Manual, 1987, ABI Inc., Foster City, Calif. In these techniques, the GLP-2 is grown from its C-terminal, resin-conjugated residue by the sequential addition of appropriately protected amino acids, using either the Fmoc or tBoc protocols, as described for instance by Orskov et al, 1989, supra.

For the incorporation of N- and/or C-protecting groups protocols is conventional to solid phase peptide synthesis methods can also be applied. For incorporation of C-terminal protecting groups, for example, synthesis of the desired peptide is typically performed using, as solid phase, a supporting resin that has been chemically modified so that cleavage from the resin results in a peptide having the desired C-terminal protecting group. To provide peptides in which the C-terminus bears a primary amino protecting group, for instance, synthesis is performed using a p-methylbenzhydrylamine, (MBHA) resin so that, when peptide synthesis is completed, treatment with hydrofluoric acid releases the desired C-terminally amidated peptide. Similarly, incorporation of an N-methylamine protecting group at the C-terminus is achieved using N methylaminoethyl-derivatized DVB resin, which upon HF treatment releases peptide bearing an N-methylamidated C-terminus. Protection of the C-terminus by esterification can also be achieved using conventional procedures. This entails use of resin/blocking group combination that permits release of side-chain protected peptide from the resin, to allow for subsequent reaction with the desired alcohol, to form the ester function. FMOC protecting groups, in combination with DVB resin derivatized with methoxyalkoxybenzyl alcohol or equivalent linker, can be used for this purpose, with cleavage from the support being effected by TFA in dichloromethane. Esterification of the suitably activated carboxyl function e.g. with DCC, can then proceed by addition of the desired alcohol, followed by deprotection and isolation of the esterified peptide product.

Incorporation of N-terminal protecting groups can be achieved while the synthesized peptide is still attached to the resin, for instance by treatment with suitable anhydride and nitrile. To incorporate an acetyl protecting group at the N-terminus, for instance, the resin-coupled peptide can be treated with 20% acetic anhydride in acetonitrile. The N-protected peptide product can then be cleaved from the resin, deprotected and subsequently isolated.

Once the desired peptide sequence has been synthesized, cleaved from the resin and fully deprotected, the peptide is then purified to ensure the recovery of a single oligopeptide having the selected amino acid sequence, Purification can be achieved using any of the standard approaches, which include reversed-phase high-pressure liquid chromatography (RP-HPLC) on alkylated silica columns, e.g. C₄-, C₈-, or C₁₈-silica. Such column fractionation is generally accomplished by running linear gradients, e.g. 10-90%, of increasing organic solvent, e.g. acetonitrile, in aqueous buffer, usually containing a small amount (e.g. 0.1%) of pairing agent such as TFA or TEA. Alternatively, ion-exchange HPLC can be employed to separate peptide species on the basis of their charge characteristics. Column fractions are collected, and those containing peptide of the desired/required purity are optionally pooled. In one embodiment of the invention, the peptide is then treated in the established manner to exchange the cleavage acid (e.g. TFA) with a pharmaceutically acceptable acid, such as acetic, hydrochloric, phosphoric, maleic, tartaric, succinic and the likes to provide a water soluble salt of the peptide.

For administration to patients, the GLP-2 compound is provided, in one aspect of the invention, in pharmaceutically acceptable form, e.g., as a preparation that is sterile-filtered e.g. through a 0.22 μm. filter, and substantially pyrogen-free. Desirably, the compound to be formulated migrates as a single or individualized peak on HPLC, exhibits uniform and authentic amino acid composition and sequence upon analysis thereof, and otherwise meets standards set by the various national bodies which regulate quality of pharmaceutical products.

For therapeutic use, the chosen compound is formulated with a carrier that is pharmaceutically acceptable and is appropriate for delivering the peptide by the chosen route of administration. Suitable pharmaceutically acceptable carriers are those used conventionally with peptide-based drugs, such as diluents, excipients and the like. Reference may be made to “Remingtons Pharmaceutical Sciences”, 17th Ed., Mack Publishing Company, Easton, Pa., 1995, for guidance on drug formulations generally. In one embodiment of the invention the compounds are formulated for administration by infusion or by injection, either subcutaneously or intravenously, and are accordingly utilized as aqueous solutions in sterile and pyrogen-free form and optionally buffered to a slightly acidic or physiological pH. Thus, the compounds may be administered in distilled water or, more desirably, in saline, buffered saline or 5% dextrose solution. Water solubility of these and other the GLP-2 may be enhanced, if desired, by incorporating a solubility enhancer, such as acetic acid.

For use in lowering circulating PTH in a mammal including a human, the present invention provides in one of its aspects a package, in the form of a sterile-filled vial or ampoule, that contains an effective PTH lowering amount of the GLP-2 compound, in either unit dose or multi-dose amounts, wherein the package incorporates a label instructing use of its contents for the reduction of PTH levels. In one embodiment of the invention, the package contains the GLP-2 and the desired carrier, as an administration-ready formulation. Alternatively, and according to another embodiment of the invention, the package provides the GLP-2 in a form, such as a lyophilized form, suitable for reconstitution in a suitable carrier, such as buffered saline.

In one embodiment, the package is a sterile-filled vial or ampoule containing an injectable solution which comprises an effective amount of GLP-2 compound dissolved in an aqueous vehicle.

As an alternative to injectable formulations, the GLP-2 compound may be formulated for administration by other routes. Oral dosage forms, such as tablets, capsules and the like, can be formulated in accordance with standard pharmaceutical practice.

In one embodiment, the GLP-2 compound is a variant resistant to cleavage by dipeptidylpeptidase-IV (DPP-IV).

In another embodiment, the GLP-2 compound is a variant which has an amino acid sequence wherein an oxidatively sensitive amino acid, is replaced with an oxidatively stable amino acid residue. In another embodiment, the oxidatively sensitive amino acid is methionine (“Met”). These variants can be more stable than a native GLP-2.

In another embodiment, the GLP-2 variant has an amino acid sequence wherein an arginine is replaced with a basic amino acid (e.g., histidine or lysine).

A GLP-2 variant or analogue may be as described in U.S. Pat. No. 6,051,557.

In specific embodiments of the invention, the GLP-2 variant may comprise a peptide having the amino acid sequence:

His-Ala-Asp-Gly-Ser-Phe-Ser-Asp-Glu-Met-Asn-Thr-
Ile-Leu-Asp-Asn-Leu-Ala-Thr-Arg-Asp-Phe-Ile-Asn-
Trp-Leu-Ile-Gln-Thr-Lys-Ile-Thr-Asp;
or
His-Ala-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp;
or
His-Gly-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp.

In a particular embodiment, GLP-2 variants have a(n):

N-terminal blocking group; and/or

N-terminal extension such as Arg or Arg-Arg; and/or

C-terminal blocking group; and/or

C-terminal extension such as Arg or Arg-Arg.

When administered to a patient, a GLP-2 compound is preferably administered as a component of a composition that optionally comprises a pharmaceutically acceptable carrier or vehicle. In a preferred embodiment, these compositions are administered orally.

Compositions for oral administration might require an enteric coating to protect the composition(s) from degradation within the gastrointestinal tract. In another example, the composition(s) can be administered in a liposomal formulation to shield the GLP-2 compound disclosed herein from degradative enzymes, facilitate the molecule's transport in the circulatory system, and effect delivery of the molecule across cell membranes to intracellular sites.

GLP-2 compounds intended for oral administration can be coated with or admixed with a material (e.g., glyceryl monostearate or glyceryl distearate) that delays disintegration or affects absorption of the GLP-2 in the gastrointestinal tract. Thus, for example, the sustained release of a GLP-2 compound can be achieved over many hours and, if necessary, the GLP-2 compound can be protected from being degraded within the gastrointestinal tract. Taking advantage of the various pH and enzymatic conditions along the gastrointestinal tract, pharmaceutical compositions for oral administration can be formulated to facilitate release of a GLP-2 compound at a particular gastrointestinal location.

Selectively permeable membranes surrounding an osmotically active driving compound are also suitable for orally administered compositions. Fluid from the environment surrounding the capsule is imbibed by the driving compound, which swells to displace the GLP-2 compound through an aperture, can provide an essentially zero order delivery profile instead of the spiked profiles of immediate release formulations. A time delay material such as, but not limited to, glycerol monostearate or glycerol stearate can also be used.

Suitable pharmaceutical carriers also include starch, glucose, lactose, sucrose, gelatin, saline, gum acacia, talc, keratin, urea, malt, rice, flour, chalk, silica gel, sodium, stearate, glycerol monostearate, talc, sodium chloride, dried skim milk, glycerol, propylene, glycol, water, and ethanol. If desired, the carrier, can also contain minor amounts of wetting or emulsifying agents, or pH buffering agents. In addition, auxiliary, stabilizing, thickening, lubricating, and colouring agents may be used. The composition can be formulated as a suppository, with traditional binders and carriers such as triglycerides.

A pharmaceutical composition comprising a GLP-2 compound can be administered via one or more routes such as, but not limited to, oral, intravenous infusion, subcutaneous injection, intramuscular, topical, depo injection, implantation, time-release mode, and intracavitary. The pharmaceutical composition is formulated to be compatible with its intended route of administration. Examples of routes of administration include parenteral, e.g., intravenous, intramuscular, intraperitoneal, intracapsular, intraspinal, intrasternal, intratumor, intranasal, epidural, intra-arterial, intraocular, intraorbital, intradermal, subcutaneous, oral (e.g., inhalation), transdermal (topical—particularly to the ears, nose, eyes, or skin), transmucosal (e.g., oral) nasal, rectal, intracerebral, intravaginal, sublingual, submucosal, and transdermal administration.

Administration can be via any route known to be effective by a physician of ordinary skill. Parenteral administration, i.e., not through the alimentary canal, can be performed by subcutaneous, intramuscular, intra-peritoneal, intratumoral, intradermal, intracapsular, intra-adipose, or intravenous injection of a dosage form into the body by means of a sterile syringe, optionally a pen-like syringe, or some other mechanical device such as an infusion pump. A further option is a composition that can be a powder or a liquid for the administration in the form of a nasal or pulmonary spray. As a still further option, the administration can be transdermally, e.g., from a patch. Compositions suitable for oral, buccal, rectal, or vaginal administration can also be provided.

In one embodiment, a pharmaceutical composition of the invention is delivered by a controlled-release system. For example, the pharmaceutical composition can be administered using intravenous infusion, an implantable osmotic pump, a transdermal patch, liposomes, or other modes of administration. In one embodiment, a pump can be used (See e.g., Langer, 1990, Science 249:1527-33; Sefton, 1987, CRC Crit. Ref. Biomed. Eng. 15:201; Buchwald et al., 1980, Surgery 88:507; Saudek et al., 1989, N. Engl. J. Med. 321:574). In another embodiment, the compound can be delivered in a vesicle, in particular a liposome (See e.g., Langer, 1990, Science 249:1527-33; Treat et al., 1989, in Liposomes in the Therapy of Infectious Disease and Cancer, Lopez-Berestein and Fidler (eds.), Liss, New York, pp. 353-65; Lopez-Berestein, ibid., pp. 317-27; International Patent Publication No. WO 91/04014; U.S. Pat. No. 4,704,355). In another embodiment, polymeric materials can be used (See e.g., Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, 1953, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In yet another embodiment, a controlled release system can be placed in proximity of the target. For example, a micropump can deliver controlled doses directly into tissue, thereby requiring only a fraction of the systemic dose (See e.g., Goodson, 1984, in Medical Applications of Controlled Release, vol. 2, pp. 115-138). In another example, a pharmaceutical composition of the invention can be formulated with a hydrogel (See, e.g., U.S. Pat. Nos. 5,702,717; 6,117,949; 6,201,072).

In one embodiment, it may be desirable to administer the pharmaceutical composition of the invention locally, i.e., to the area in need of treatment. Local administration can be achieved, for example, by local infusion during surgery, topical application (e.g., in conjunction with a wound dressing after surgery), injection, catheter, suppository, or implant. An implant can be of a porous, non-porous, or gelatinous material, including membranes, such as sialastic membranes, or fibres.

In certain embodiments, it may be desirable to introduce the GLP-2 into the central nervous system by any suitable route, including intraventricular, intrathecal, and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

In one embodiment, the invention provides for the treatment of a patient using implanted cells that have been regenerated or stimulated to proliferate in vitro or in vivo prior to reimplantation or transplantation into a recipient. Conditioning of the cells ex vivo can be achieved simply by growing the cells or tissue to be transplanted in a medium that has been supplemented with a growth-promoting amount of the combinations and is otherwise appropriate for culturing of those cells. The cells can, after an appropriate conditioning period, then be implanted either directly into the patient or can be encapsulated using established cell encapsulation technology, and then implanted.

The skilled artisan can appreciate the specific advantages and disadvantages to be considered in choosing a mode of administration. Multiple modes of administration are encompassed by the invention. For example, a GLP-2 compound of the invention can be administered by subcutaneous injection, whereas another therapeutic agent can be administered by intravenous infusion. Moreover, administration of one or more kinds of GLP-2 compound, with or without other therapeutic agents, can occur simultaneously (i.e., co-administration) or sequentially. In another embodiment, the periods of administration of a GLP-2 compound, with or without other therapeutic agents can overlap. For example a GLP-2 compound can be administered for 7 days and another therapeutic agent can be introduced beginning on the fifth day of treatment. Treatment with the other therapeutic agent can continue beyond the 7-day GLP-2 treatment.

A pharmaceutical composition of a GLP-2 compound can be administered before, during, and/or after the administration of one or more therapeutic agents. A pharmaceutical composition of the invention can be administered in the morning, afternoon, evening, or diurnally. In one embodiment, the pharmaceutical composition is administered at particular phases of the circadian rhythm. In a specific embodiment, the pharmaceutical composition is administered in the morning. In another specific embodiment, the pharmaceutical composition is administered at an artificially induced circadian state.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (See e.g., U.S. Pat. No. 5,698,155). Other examples of suitable pharmaceutical carriers are described in Remington's Pharmaceutical Sciences, Alfonso R. Gennaro ed., Mack Publishing Co. Easton, Pa., 19^{th} ed., 1995, pp. 1447 to 1676, incorporated herein by reference.

Accordingly, the pharmaceutical compositions herein described can be in the form of oral tablets, capsules, elixirs, syrups and the like.

For instance, for oral administration in the form of a tablet or capsule, the active drug component can be combined with an oral, non-toxic, pharmaceutically acceptable, inert carrier such as, but not limited to, lactose, starch, sucrose, glucose, methyl cellulose, magnesium stearate, dicalcium phosphate, calcium sulfate, mannitol, and sorbitol. For oral administration in liquid form, the oral drug components can be combined with any oral, non-toxic, pharmaceutically acceptable carrier such as, but not limited to, ethanol, glycerol, and water. Moreover, suitable binders, lubricants, disintegrating agents and coloring agents can also be incorporated into the mixture. Suitable binders include, but are not limited to, starch, gelatin, natural sugars (e.g., glucose, beta-lactose), corn sweeteners, natural and synthetic gums (e.g., acacia, tragacanth, sodium alginate), carboxymethylcellulose, polyethylene glycol, and waxes. Lubricants useful for an orally administered drug, include, but are not limited to, sodium oleate, sodium stearate, magnesium stearate, sodium benzoate, sodium acetate, and sodium chloride. Disintegrators include, but are not limited to, starch, methyl cellulose, agar, bentonite, and xanthan gum.

Pharmaceutical compositions adapted for oral administration can be provided, for example, as capsules or tablets; as powders or granules; as solutions, syrups or suspensions (in aqueous or non-aqueous liquids); as edible foams or whips; or as emulsions. For oral administration in the form of a tablet or capsule, the active drug component can be combined with an oral, non-toxic, pharmaceutically acceptable, inert carrier such as, but not limited to, lactose, starch, sucrose, glucose, methyl cellulose, magnesium stearate, dicalcium phosphate, magnesium carbonate, stearic acid or salts thereof, calcium sulfate, mannitol, and sorbitol. For oral administration in the form of a soft gelatine capsule, the active drug component can be combined with an oral, non-toxic, pharmaceutically acceptable, inert carrier such as, but not limited to, vegetable oils, waxes, fats, semi-solid, and liquid polyols. For oral administration in liquid form, the oral drug components can be combined with any oral, non-toxic, pharmaceutically acceptable carrier such as, but not limited to, ethanol, glycerol, polyols, and water. Moreover, suitable binders, lubricants, disintegrating agents and coloring agents can also be incorporated into the mixture. Suitable binders include, but are not limited to, starch, gelatin, natural sugars (e.g., glucose, beta-lactose), corn sweeteners, natural and synthetic gums (e.g., acacia, tragacanth, sodium alginate), carboxymethylcellulose, polyethylene glycol, and waxes. Lubricants useful for an orally administered drug, include, but are not limited to, sodium oleate, sodium stearate, magnesium stearate, sodium benzoate, sodium acetate, and sodium chloride. Disintegrators include, but are not limited to, starch, methyl cellulose, agar, bentonite, and xanthan gum.

Orally administered compositions may contain one or more agents, for example, sweetening agents such as, but not limited to, fructose, ASPARTAME and saccharin. Orally administered compositions may also contain flavoring agents such as, but not limited to, peppermint, oil of wintergreen, and cherry. Orally administered compositions may also contain colouring agents and/or preserving agents.

The GLP-2 compound can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. A variety of cationic lipids can be used in accordance with the invention including, but not limited to, N-(1(2,3-dioleyloxy)propyl)-N,N,N-trimethylammonium chloride (“DOTMA”) and diolesylphosphotidylethanolamine (“DOPE”). Such compositions suit the mode of administration.

GLP-2 compounds can also be delivered by the use of monoclonal antibodies as individual carriers to which the GLP-2 compounds can be coupled. The GLP-2 compounds can also be coupled with soluble polymers as targetable drug carriers. Such polymers can include polyvinylpyrrolidone, pyran copolymer, polyhydroxypropylmethacrylamide-phenol, polyhydroxyethylaspartamide-phenol, or polyethyleneoxide-polylysine substituted with palmitoyl residues. Furthermore, the GLP-2 compounds can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

Pharmaceutical compositions adapted for parenteral administration include, but are not limited to, aqueous and non-aqueous sterile injectable solutions or suspensions, which can contain antioxidants, buffers, bacteriostats and solutes that render the pharmaceutical compositions substantially isotonic with the blood of an intended recipient. Other components that can be present in such pharmaceutical compositions include water, alcohols, polyols, glycerine and vegetable oils, for example. Compositions adapted for parenteral administration can be presented in unit-dose or multi-dose containers (e.g., sealed ampoules and vials), and can be stored in a freeze-dried (i.e., lyophilized) condition requiring the addition of a sterile liquid carrier (e.g., sterile saline solution for injections) immediately prior to use. Extemporaneous injection solutions and suspensions can be prepared from sterile powders, granules and tablets.

Pharmaceutical compositions adapted for transdermal administration can be provided as discrete patches intended to remain in intimate contact with the epidermis for a prolonged period of time. Pharmaceutical compositions adapted for topical administration can be provided as, for example, ointments, creams, suspensions, lotions, powders, solutions, pastes, gels, sprays, aerosols or oils. A topical ointment or cream is preferably used for topical administration to the skin, mouth, eye or other external tissues. When formulated in an ointment, the active ingredient can be employed with either a paraffinic or a water-miscible ointment base. Alternatively, the active ingredient can be formulated in a cream with an oil-in-water base or a water-in-oil base.

Pharmaceutical compositions adapted for topical administration to the eye include, for example, eye drops or injectable pharmaceutical compositions. In these pharmaceutical compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilles and mouthwashes.

Pharmaceutical compositions adapted for nasal administration can comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, pharmaceutical compositions adopted for nasal administration can comprise liquid carriers such as, for example, nasal sprays or nasal drops. These pharmaceutical compositions can comprise aqueous or oil solutions of a GLP-2 molecule. Compositions for administration by inhalation can be supplied in specially adapted devices including, but not limited to, pressurized aerosols, nebulizers or insufflators, which can be constructed so as to provide predetermined dosages of the GLP-2 compound.

Pharmaceutical compositions adapted for rectal administration can be provided as suppositories or enemas. Pharmaceutical compositions adapted for vaginal administration can be provided, for example, as pessaries, tampons, creams, gels, pastes, foams or spray formulations.

Suppositories generally contain active ingredients in the range of 0.5% to 10% by weight. Oral formulations preferably contain 10% to 95% active ingredient by weight. In a preferred embodiment, the composition is formulated in accordance with routine procedures as a pharmaceutical composition adapted for intratumoral injection, implantation, subcutaneous injection, or intravenous administration to humans.

Typically, pharmaceutical compositions for injection or intravenous administration are solutions in sterile isotonic aqueous buffer. Where necessary, the composition can also include a solubilising agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachet indicating the quantity of active agent.

Where the composition is to be administered by infusion, it can be dispensed with an infusion bottle, bag, or other acceptable container, containing sterile pharmaceutical grade water, saline, or other acceptable diluents. Where the composition is administered by injection, an ampoule of sterile water for injection or saline can be provided so that the ingredients may be mixed prior to administration.

Methods and compositions for administration disclosed in relation to GLP-2 above are applicable equally to combination therapies disclosed herein.

The GLP-2 and optionally another therapeutic agent are administered at an effective dose. The dosing and regimen most appropriate for patient treatment will vary with the disease or condition to be treated, and in accordance with the patient's weight and with other parameters.

An effective dosage and treatment protocol can be determined by conventional means, comprising the steps of starting with a low dose in laboratory animals, increasing the dosage while monitoring the effects (e.g., histology, disease activity scores), and systematically varying the dosage regimen. Several factors may be taken into consideration by a clinician when determining an optimal dosage for a given patient. Primary among these is the amount of GLP-2 molecule normally circulating in the plasma, which, in the case of a GLP-2 peptide, is approximately 150 pmol/ml in the resting state, and rising to approximately 225 pmol/ml after nutrient ingestion for healthy adult humans (Orskov and Holst, 1987, Scand J. Clin. Lab. Invest. 47:165). Additional factors include, but are not limited to, the size of the patient, the age of the patient, the general condition of the patient, the particular disease being treated, the severity of the disease, the presence of other drugs in the patient, and the in vivo activity of the GLP-2 molecule.

Trial dosages would be chosen after consideration of the results of animal studies and the clinical literature. A person of ordinary skill in the art can appreciate that information such as binding constants and Ki derived from in vitro GLP-2 binding competition assays may also be used in calculating dosages.

A typical effective human dose of a GLP-2 compound would be from about 10 μg/kg body weight/day to about 10 mg/kg/day, preferably from about 50 μg/kg/day to about 5 mg/kg/day, and most preferably about 100 μg/kg/day to 1 mg/kg/day. As variants and analogues of GLP-2 disclosed herein can be 2 to 100 times more potent than naturally occurring counterparts, a typical effective dose of such a GLP-2 variant or analog can be lower, for example, from about 100 ng/kg body weight/day to 1 mg/kg/day, preferably 1 μg/kg/day to 500 μg/kg/day, and even more preferably 1 μg/kg/day to 100 μg/kg/day.

In another embodiment, the effective dose of a GLP-2 is less than 10 μg/kg/day. In yet another embodiment the effective dose of a GLP-2 compound is greater than 10 mg/kg/day.

The specific dosage for a particular patient, of course, has to be adjusted to the degree of response, the route of administration, the patients weight, and the patient's general condition, and is finally dependent upon the judgment of the treating physician.

The effectiveness of the methods of treatment of the invention on a patient can be evaluated by, for example, determining the level of plasma PTH. Thus, changes in the PTH level after GLP-2 administration can monitor treatment effectiveness.

### BRIEF DESCRIPTION OF THE DRAWINGS

FIG. 1 shows plasma PTH levels measured in Example 1;

FIG. 2 shows plasma PTH levels measured in Example 2;

FIG. 3 shows plasma PTH levels measured in Example 3; and

FIG. 3 shows further plasma PTH levels measured in Example 3.

The present invention may be better understood by reference to the following non-limiting Examples, which are provided only as exemplary of the invention. The following examples are presented to more fully illustrate the preferred embodiments of the invention. They should in no way be construed, however, as limiting the broader scope of the invention.

### EXAMPLES

The GLP-2 used in all the examples was human GLP-2 1-34.

### Example 1

### GLP-2 Results After Morning Administration for Plasma PTH in Fasting Subject

The study was a randomized, block-stratified, placebo-controlled, parallel study. A total of 60 subjects of postmenopausal women were included and allocated to one of five groups each including 12 subjects receiving one dose of 100 μg, 200 μg, 400 μg, 800 μg GLP-2, or placebo.

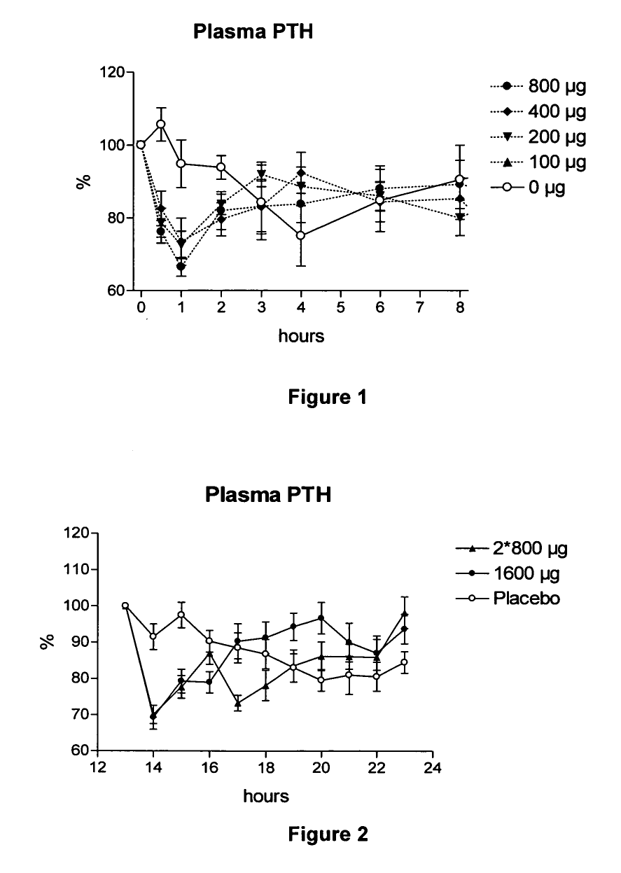

On the day of treatment with the study medication, the subjects met at the study site after an overnight fast. The study medication was administered at 9 am (0 hrs) and the subjects remained fasting for another 8 hrs, thus at 5 pm the fasting period ended. Results are shown in FIG. 1.

Administration of GLP-2 is seen to produce a dose dependent rapid decrease in PTH which gradually abates over an 8 hour period.

### Example 2

### GLP-2 Results After Bedtime Administration for Plasma PTH in Non-Fasting Subject

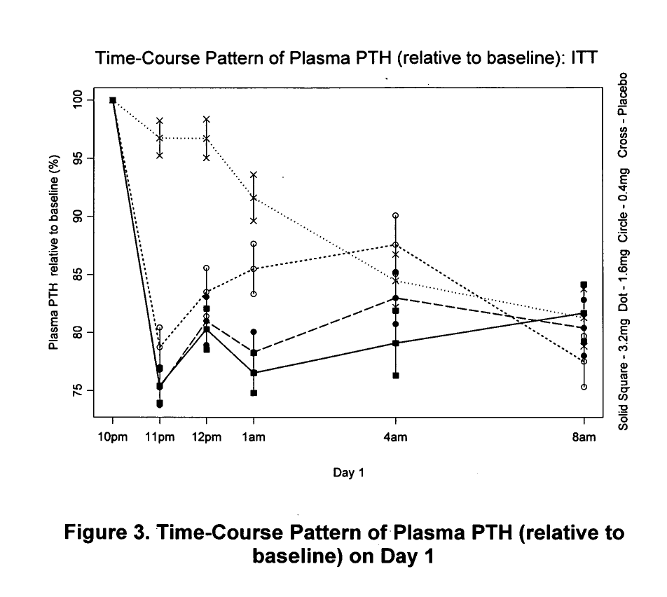

The study was a randomized, block-stratified, placebo-controlled, parallel study. A total of 44 postmenopausal women were included and randomly assigned to treatment with either 1600 μg GLP-2 (n=14), 800 μg GLP-2 twice (administered 3 hrs apart) (n=15), or placebo, i.e. saline (n=15). Results are shown in FIG. 2. All subjects received 2 injections one at 10 pm (13 hours) and one 3 hrs later at 1 am (16 hours). Thus, the placebo group was given two injections of saline and the 2×800 μg group two injections of 800 μg GLP-2, while the 1600 μg group received one injection of 1600 μg GLP-2 and one injection of saline.

On the day of treatment with the study medication, blood samples for efficacy measurement was taken from 10 pm every hour until 8 am.

It can be seen that two injections of 800 μg produced a greater area difference between the treatment and placebo curves.

### Example 3

### GLP-2 Results for Plasma PTH at Day-1 and Day-120 After Bedtime Administration in 120 Days

The study was a randomised, double-blinded, placebo-controlled, parallel group and dose-ranging study in 160 postmenopausal women with osteopenia (BMD T-score, −2.5≦T≦−1.0) at one or more of the regions: the lumbar spine, femoral neck or total hip. Forty (40) subjects were randomised to each treatment group to receive treatment with daily doses of 0.4 mg, 1.6 mg or 3.2 mg of GLP-2 plus calcium (800-1000 mg/day) and vitamin D (400-800 IU/day), or placebo plus calcium (800-1000 mg/day) and vitamin D (400-800 IU/day) for 120 days.

The treatment involved a daily injection of the study medication at bedtime (10:00 pm±1 hour) for 120 consecutive days.

Pharmacokinetic/dynamic sampling during treatment Day 1 and 120 included blood and urine samples collected at the time points: −1 hour, 0 min, 1 hour, 2 hours, 3 hours, 6 hours and 10 hours in connection with the injection of the study medication for the assessment of efficacy parameters and pharmacokinetic/dynamic parameters of subcutaneous injections of GLP-2.

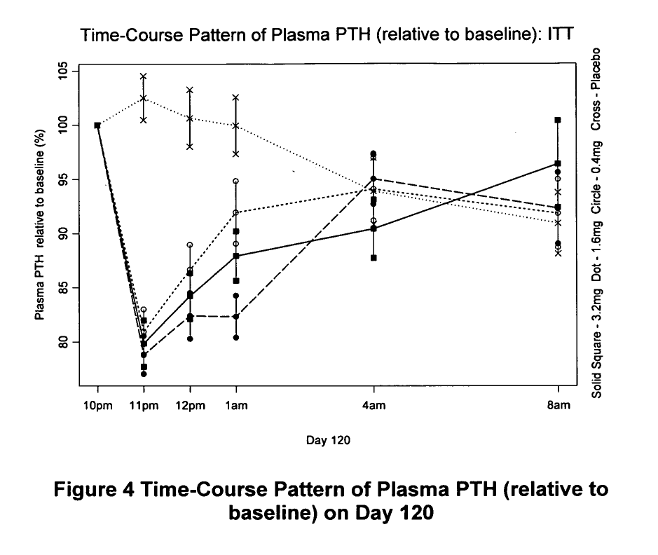

Results are shown in FIGS. 3 and 4 for days 1 and 120. It is seen that the effect of GLP-2 on PTH over the treatment period remains consistent.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

1. A method of acutely reducing the plasma level of PTH of a patient having an elevated PTH comprising the administration of a pharmaceutical composition comprising a GLP-2, or a variant, an analogue, or derivative of GLP-2 having the ability to bind and activate a GLP-2 receptor.

2. A method as claimed in claim 1, wherein said elevated level of PTH is primary, secondary, or tertiary hyperparathyroidism.

3. A method as claimed in claim 1, wherein said elevated level of PTH is hyperparathyroidism secondary to gastrointestinal bypass surgery.

4. A method as claimed in claim 1, wherein said elevated PTH is secondary to dietary calcium malabsorption.

5. A method as claimed in claim 1, wherein said elevated level of PTH is hyperparathyroidism linked to obesity.

6. A method as claimed in claim 1, comprising administration of GLP-2 1-34.


SEQUENCE LISTING (23 sequences)

SEQ ID NO: 1 - protein, 35 aa, Homo sapiens
HADGSFSDEMNTILDNLAARDFINWLIQTKITDRK

SEQ ID NO: 2 - protein, 35 aa, Sus sp.
HADGSFSDEMNTVLDNLATRDFINWLLHTKITDSL

SEQ ID NO: 3 - protein, 35 aa, Rattus sp.
HADGSFSDEMNTILDNLATRDFINWLIQTKITDKK

SEQ ID NO: 4 - protein, 35 aa, Unknown
  Description of Unknown Hamster GLP-2 sequence
HADGSFSDEMNTILDSLATRDFINWLIQTKITDKK

SEQ ID NO: 5 - protein, 35 aa, Unknown; identical to SEQ ID NO: 1
  Description of Unknown Anglerfish GLP-2 sequence

SEQ ID NO: 6 - protein, 35 aa, Cavia sp.
HADGSFSDEMNTILDNLATRDFINWLIQTKITDRK

SEQ ID NO: 7 - protein, 35 aa, Unknown
  Description of Unknown Bovine GLP-2 sequence
HADGSFSDEMNTVLDSLATRDFINWLLQTKITDRK

SEQ ID NO: 8 - protein, 35 aa, Mus sp.
HADGSFSDEMSTILDNLATRDFINWLIQTKITDRK

SEQ ID NO: 9 - protein, 35 aa, Canis sp.
HADGSFSDEMNTVLDTLATRDFINWLLQTKITDRK

SEQ ID NO: 10 - protein, 35 aa, Octodon degus
HADGSFSDEMNTVLDHLATKDFINWLIQTKITDRK

SEQ ID NO: 11 - protein, 35 aa, Unknown
  Description of Unknown Salamander GLP-2 sequence
HADGSFTSDINKVLDTIAAKEFLNWLISTKVTERK

SEQ ID NO: 12 - protein, 37 aa, Artificial Sequence
  Description of Artificial Sequence Synthetic polypeptide
XXHADGSFSDEMNTXLAXLAXRDFINWLXXTKITDXX

SEQ ID NO: 13 - protein, 33 aa, Artificial Sequence
  Description of Artificial Sequence Synthetic polypeptide
HADGSFSDEMNTILDNLATRDFINWLIQTKITD

SEQ ID NO: 14 - protein, 37 aa, Artificial Sequence
  Description of Artificial Sequence Synthetic polypeptide
XXHADGSFSDEMNTXLDXLAXRDFINWLXXTKITDXX

SEQ ID NO: 15 - protein, 37 aa, Artificial Sequence; identical to SEQ ID NO: 14
  Description of Artificial Sequence Synthetic polypeptide

SEQ ID NO: 16 - protein, 37 aa, Artificial Sequence
  Description of Artificial Sequence Synthetic polypeptide
XXXXXXSFSDXXXXXLDNLAXXDFXXWLIQTKXXXXX

SEQ ID NO: 17 - protein, 5 aa, Artificial Sequence
  Description of Artificial Sequence Synthetic peptide
EXNTI

SEQ ID NO: 18 - protein, 4 aa, Artificial Sequence
  Description of Artificial Sequence Synthetic peptide
YSKY

SEQ ID NO: 19 - protein, 37 aa, Artificial Sequence
  Description of Artificial Sequence Synthetic polypeptide
XXXXXXXFXDXXXXXLXXLAXXDXXXWLXXXKXXXXX

SEQ ID NO: 20 - protein, 33 aa, Artificial Sequence
  Description of Artificial Sequence Synthetic polypeptide
XXXXXFXDXXXXXLXXLAXXDXXXWLXXXKXXX

SEQ ID NO: 21 - protein, 37 aa, Artificial Sequence
  Description of Artificial Sequence Synthetic polypeptide
XXHADGSFSDEMNTILDNLAXRDFINWLIQTKITDXX

SEQ ID NO: 22 - protein, 33 aa, Artificial Sequence
  Description of Artificial Sequence Synthetic polypeptide
HADGSFSDEMNTILDNLAARDFINWLIQTKITD

SEQ ID NO: 23 - protein, 33 aa, Artificial Sequence
  Description of Artificial Sequence Synthetic polypeptide
HGDGSFSDEMNTILDNLAARDFINWLIQTKITD